(19) United States
(12) Patent Application Publication
(10) Pub. No.: US 2002/0044942 A1
(43) Pub. Date: Apr. 18, 2002

(54) Transfer factor composition and process for producing same

(75) Inventor: Minter H. Dopson, Aiken, SC (US)

Correspondence Address: Maria Reichmanis, P.O. Box 3306, Aiken, SC, 29802, US

(73) Assignee: Chisolm Biological Laboratory, LLC

(21) Appl. No.: 09/954,961

(22) Filed: Sep. 18, 2001

Related U.S. Application Data
(60) Provisional application No. 60/233,400, filed on Sep. 18, 2000.

(51) Int. Cl. A61K 39/00; C07K 16/00

(52) U.S. Cl. 424/184.1; 530/387.1

(57) ABSTRACT

A process for producing transfer factor from the eggs of birds, compositions containing transfer factor so produced, and methods for using the compositions. Transfer factor is produced by administering a selected antigen to adult female birds (preferably domestic chickens), waiting for a sufficient period of time for the birds to develop immunity against the antigen, then recovering the antigen-specific transfer factor from the eggs laid by the birds. The transfer factor may be incorporated into topical compositions, edible compositions, dietary supplements, and pharmaceutical or veterinary compositions for the prevention or treatment of a disease associated with the selected antigen(s).

### BACKGROUND OF THE INVENTION

1. Field of the Invention

The present invention relates to a process for producing a transfer factor-containing composition. In particular, the present invention relates to a process for producing a transfer factor-containing composition from the eggs of birds, a composition produced by the process, products containing the composition, and methods for using the composition and the products.

2. Discussion of Background

When an organism contracts a bacterial, parasitic or viral infection, its immune system combats the infection with white blood cells and antibodies. This response is frequently sufficient to overcome the infection and restore the organism to its normal state. In the case of massive infections or in organisms with deficient immune systems, however, this naturally-occurring immune response is not sufficient to combat the infection.

Vaccinations are helpful in immunizing humans and other animals against some infectious agents: vaccination can be viewed as “teaching” the immune system to recognize an infectious agent in advance, so the organism is ready to manufacture the appropriate antibodies if and when it is actually infected. Antibiotics are another useful treatment modality for combating infections; however, due to the widespread overuse of antibiotics, many pathogens are becoming increasingly antibiotic-resistant.

Transfer factor (also known as “immune factor” or “DLE” (dialyzable leukocyte extract”)) offers another line of defense against infection, since it contains protein immunomodulators that transfer the ability to express cell-mediated immunity from donors to recipients. In that sense, administering transfer factor to a recipient is analogous to vaccination. Some of the immune reaction in humans and other animals is due to the action of antibodies which combine chemically with, and neutralize, the corresponding antigens. However, systemic immunity is also mediated by transfer factor, which can be viewed as a carrier of the cellular immune memory: the lymphocytes of a normal adult contain transfer factors corresponding to every bacterial, viral, or other antigen with which he or she has been in contact. Every antigen has a corresponding, separate transfer factor, thus, there are believed to be as many different transfer factors as there are antigens. In some cases, treatment with transfer factor may help activate or enhance the recipient's immune system to help fight an infection; in others, treatment may optimize the recipient's immune system, helping suppress an overly active immune system or stimulating non-specific cell-mediated immunity in a sluggish immune system.

At least three different forms of transfer factor are known: excreted transfer factor (“TFe”), which is released from transfer factor-containing cells and may be collected from extra-cellular fluid, pre-excreted transfer factor (“TFpre”) which occurs within the cell or on the cell surface, and is believed to be released as TFe; internal transfer factor (“TFi”) is found within the cell, and is believed to differ in chemical structure from TFe and TFpre. Transfer factors are dialyzable materials having molecular weights of approximately 3000-6,000 (i.e., transfer factors are smaller than typical antibodies). They are substantially non-antigenic (i.e., do not induce immune reactions), do not transfer antibody-mediated responses, and do not induce antibody production in the recipient. Transfer factors have been used to treat deficiencies of cellular immunity, and have also been used in the treatment of a variety of disease conditions (candidiasis, measles, mumps, chickenpox, and so forth).

Antigen-specific TFe may be produced from the milk or colostrum of lactating mammals. Alternatively, antigen-specific transfer factor may be produced from the mammary tissue of animals that have been previously sensitized to a selected antigen in such a manner as to express delayed-type hypersensitivity or other cell-mediated responses to that antigen. Transfer factor can also be obtained from the blood serum leukocytes and lymph node lymphocytes of sensitized animals. At present, colostrum from cows or goats is the preferred source material for TFe.

To obtain transfer factor, a suitable mammal is immunized against a selected antigen or antigens (bacteria, viruses, rickettsiae, fungi, protozoa, etc.). After a sufficient period of time has elapsed for the mammal to respond to the antigen, its colostrum or milk is collected. Cows, for example, are immunized between the fifth and seventh month of gestation with one or more commercially-available veterinary vaccines. The transfer-factor-containing milk or colostrum may be used directly, or may be treated by filtration or dialysis to recover the transfer factor.

Alternatively, transfer factor is isolated from leukocytes collected from whole blood, spleen or lymph node tissue. Transfer factor is used in dietary supplements, topical compositions, pharmaceutical compositions, and veterinary compositions to help treat conditions associated with the selected antigen or antigens. Transfer factor is also used to condition or optimize the recipient's immune system.

Many processes for making transfer factor are available, typically starting with milk, colostrum, or other tissue obtained from suitable animals, and relying on one or more of the following techniques to recover transfer factor from the starting material: centrifugation, lyophilization, dialysis, and cell disruption by alternately freezing and thawing. For example, Kirkpatrick describes the production of transfer factor from the lymphoid cells of humans and other animals, and its characteristics and uses (U.S. Pat. Nos. 5,883,224, 5,840,700, and 5,470,835). The transfer factor is produced as follows: a transfer-factor-containing sample is contacted to an immobilized antigen to which the transfer factor binds under conditions favoring binding to the antigen to form a transfer factor:antigen complex. The antigen-specific transfer factor is then separated from the complex, passed through an HPLC column, eluted, passed through a gel filtration HPLC column, and eluted.

Wilson, et al. (U.S. Pat. No. 4,816,563) disclose a method for obtaining a cell-free fluid containing excreted transfer factor (TFe) from material secreted by the mammary glands of lactating animals; other sources of transfer factor include separated colostral cells and mammary tissue. Animals are injected with a selected antigen (bacteria, fungi, virus, etc.), transfer factor specific for the antigen, or are exposed to the antigen before commencing lactation. After commencement of lactation, the animals' milk or colostrum is collected, cells and cell debris are separated, and the resulting cell-free supernatant is collected and (optionally) purified and concentrated.

Warren (U.S. Pat. No. 4,435,384) describes a topical composition that contains transfer factor, a penetrant (dimethyl sulfoxide or low molecular weight dextran), and a carrier. The transfer factor is obtained from the lymphocytes of a donor having no history of recurrent infection with the herpes virus, and is produced by a process that includes the following steps: incubating the separated plasma, centrifuging, washing the separated cells with 0.5N saline, reconstituting with normal saline, alternately freezing and thawing for a total of five times, and centrifuging to separate the supernatant fluid containing transfer factor.

Jeter (U.S. Pat. No. 4,132,776) prepares an edible composition by adding a carrier (saline) to transfer factor. The transfer factor is prepared by suspending separated buffy coat cells in distilled water, disrupting the cells by freezing and thawing, and removing cellular debris by centrifugation. The resulting clear supernatant liquid is dialyzed, lyophilized, and stored at −20° C. until used.

Goust, et al. (U.S. Pat. No. 4,001,080) produce transfer factor by in vitro culture of lymphoblastoid cell lines in the presence of transfer factor which acts as a catalyst for the production of additional transfer factor by the cells. The transfer factor so produced is extracted from the cells or recovered by dialysis of the culture medium.

Spitler (U.S. Pat. No. 3,991,182) treats human patients with immune deficiency diseases (Wiskott-Aldrich syndrome, Swiss type agamma-globulinemia) by injecting them with transfer factor obtained from the leukocytes (i.e., white blood cells) of healthy donors. The transfer factor is prepared by adding an EDTA type anticoagulant to a blood sample, suspending in saline, alternately freezing and thawing the suspension, lysing by incubation in the presence of magnesium and DNase, dialyzing against distilled water, separating and lyophilizing the dialysate, reconstituting with distilled water, and filtering.

Yoshida, et al. (U.S. Pat. No. 4,594,245) produce augmenting factors (including immunoglobulins) from cultured human lymphocytes. The lymphocytes are recovered from various organs and peripheral blood, cultured, and separated into two groups: those that adhere to nylon wool and those that don't. The nylon-wool-adherent lymphocytes are cultured, then the culture medium is dialyzed to obtain a fluid that contains the desired factors. The disclosures of the above-referenced patents are incorporated herein by reference.

Despite the many known processes for producing transfer factor, none lends itself to the large-scale production of antigen-specific transfer factors having consistent, reproducible properties: lymphocytes and other cells are difficult to maintain in cell culture for extended periods of time, and animals such as cows and goats are expensive to maintain. There is a continuing need for a reliable, costeffective process that does not depend on cell culture or on the availability of cows, goats, or other large mammals.

### SUMMARY OF THE INVENTION

According to its major aspects and broadly stated, the present invention includes a process for producing a transfer factor-containing composition from the eggs of birds, the transfer-factor-containing composition so produced, products containing the composition, and methods for using the composition and the products. The invention is based on the surprising discovery that the eggs of appropriately treated birds, particularly the eggs of domestic chickens (Gallus gallus) contain useful amounts of transfer factor. The composition is produced by administering at least one selected antigen to adult female chickens (hens) or other suitable female birds, waiting for a sufficient period of time for the birds to develop immunity to the antigen, then recovering antigen-specific transfer factor from eggs laid by the birds.

An important feature of the present invention is the use of bird eggs rather than mammalian tissue (lymphocytes, milk, colostrum, etc.) as the starting material. The eggs of chickens, ducks, emus, geese, ostriches, turkeys, and other egg-laying birds are broadly suitable for the practice of the invention. However, the eggs of domestic chickens are preferred due to the low cost, straightforward maintenance requirements, and ready availability of these birds. The breeds which produce relatively large eggs in proportion to their body size are particularly useful (for purposes of this specification, the terms “breed” and “variety” are used interchangeably). Another advantage of using chickens and chicken eggs is that, like certain strains of laboratory animals widely used in medical research, the varieties of chickens used in commercial poultry farming are genetically standardized (that is, chickens of any selected variety are quite uniform in both genotype and phenotype). Transfer factor obtained from such standardized varieties is expected to be relatively uniform in concentration and potency.

Another feature of the present invention is the process for producing the composition from eggs. Eggs laid by the birds after they develop immunity to the antigen (or antigens) are collected and treated by standard procedures (cleaning, storage, and so forth), and the composition is produced from the egg whites, yolks, or the combined whites and yolks. In one embodiment of the invention, the egg whites and yolks are separated from the shells by any convenient process, and the shells are discarded. The yolks (alternatively, the whites, or the combined whites and yolks) are treated to substantially remove cells and cell debris, leaving a substantially cell-free fluid that contains the desired transfer factors. Useful techniques include filtration, ultrafiltration, centrifugation, dialysis, microdialysis, HPLC (high performance liquid chromatography), precipitation, lyophilization, cell disruption by repeated freezing and thawing, and combinations thereof. If desired, the transfer factor may be further concentrated, purified, or both.

In another embodiment of the invention, a selected amount of egg yolks, in the natural state, is added to the composition, preferably with an effective amount of sodium chlorate. The natural constituents in egg yolk are believed to help preserve the bioactivity of transfer factor when it passes through the digestive tract (particularly the acidic conditions of the stomach), thereby resulting in higher bioactivity and actual quantities of effective transfer factor in the gut. Sodium chlorate help ensure protection against bacteria such as salmonella and E. coli that may be present in the egg yolks.

In still another embodiment of the invention, an effective amount of sodium chlorate is administered to the hens. Eggs laid a sufficient period of time after ingestion of the sodium chlorate are believed to contain fewer potentially-harmful bacteria (salmonella, E. coli, etc.).

Still another feature of the present invention is the ability to produce a composition containing transfer factor specific to virtually any selected antigen (or antigens), simply by immunizing the hens with that antigen. Suitable antigens include any of a variety of bacteria, viruses, rickettsiae, fungi, protozoa, and associated vaccines.

Yet another feature of the present invention is the ability to use the composition made by the above-described process in a variety of products. The composition may be incorporated into edible products, including but not limited to nutraceuticals, dietary supplements, and pharmaceutical or veterinary compositions for the prevention or treatment of a disease associated with the selected antigen(s). The transfer-containing composition may be added to products intended for topical use (lotions, creams, cleansers, toners, etc.); such products may also include one or more of the following: nontoxic carriers, cleansing agents, humectants, emollients, penetrants such as DMSO, stabilizers, biocompatible perfumes and coloring agents, and other useful constituents..

Another feature of the present invention is the use of the composition, and products containing the composition, in the treatment and prevention of disease. The composition is broadly useful in activating, stimulating, or enhancing the immune system, since transfer factor can help confer immunity to the recipient against many different antigens and the diseases associated therewith. For example, patients suffering from septicemia, sinusitis, influenza, diabetes, bronchitis, autism, infertility, the common cold, herpes, bronchitis, measles, mumps, systemic lupus erythematosus (SLE), fibromyalgia, post-herpetic neuralgia, chronic fatigue syndrome, HIV, hepatitis, multiple sclerosis, and cancer have been treated with transfer factor. The composition may also be useful as a general immune system toner or optimizer, since administration of transfer factor is known to stimulate sluggish or compromised immune systems, and, conversely, suppress overly-active immune systems.

Other features and advantages of the present invention will be apparent to those skilled in the art from a careful reading of the Detailed Description of Preferred Embodiments presented below and accompanied by the drawings.

### BRIEF DESCRIPTION OF THE DRAWINGS

In the drawings,

FIG. 1 is a flow chart illustrating a process for producing antigen-specific transfer factor according to a preferred embodiment of the present invention.

### DETAILED DESCRIPTION OF PREFERRED EMBODIMENTS

In the following detailed description of the invention, the drawings are intended to be read together with the specification, and are to be considered a portion of the entire written description of this invention as required by 35 U.S.C. §112.

The present invention is based on the surprising discovery that the eggs of avian species contain useful amounts of transfer factor. While the eggs of all egg-laying birds (ducks, geese, emus, ostriches, turkeys, etc.) are broadly suitable for use with the invention, the eggs of domestic chickens of the family Phasianidae (Gallus gallus, also known as gallinaceous or galliforme birds) are preferred due to the low cost, fast maturation, easy maintenance requirements, and ready availability of these birds. Particularly useful are the breeds which produce eggs that are relatively large in proportion to their body size, including but not limited to American (Rhode Island Red, N.H., Plymouth Rock, etc.) and Mediterranean (Ancona, Andalusian, Minorca, Leghorn) breeds.

A bird egg consists of the protoplasm from which the animal eventually develops, a much larger amount of nutritive material (the yolk or deutoplasm), an albuminous mass (the egg white), a membrane, and a shell mainly composed of calcium carbonate. Both the albumin and the yolk contain transfer factor; however, much of the transfer factor is in the yolk.

The yolk delivers high-density nutrition to the developing embryo during gestation. It also confers general passive immunity (both to the embryo and the infant chick) in the form of antibodies to pathogens and other antigens that were present in the mother's environment when the egg was formed. When suitably treated (as will be described below), the yolk may also contain specific and nonspecific, physiologically active factors that stimulate the immune system of the embryo.

Chickens that are immunized with human pathogens produce antibodies specific to those pathogens in addition in addition to nonspecific factors that benefit the circulatory and immune systems, the digestive tract, and the joints. Immunized birds produce eggs that provide concentrated sources of these antibodies, and are also surprisingly rich in transfer factor.

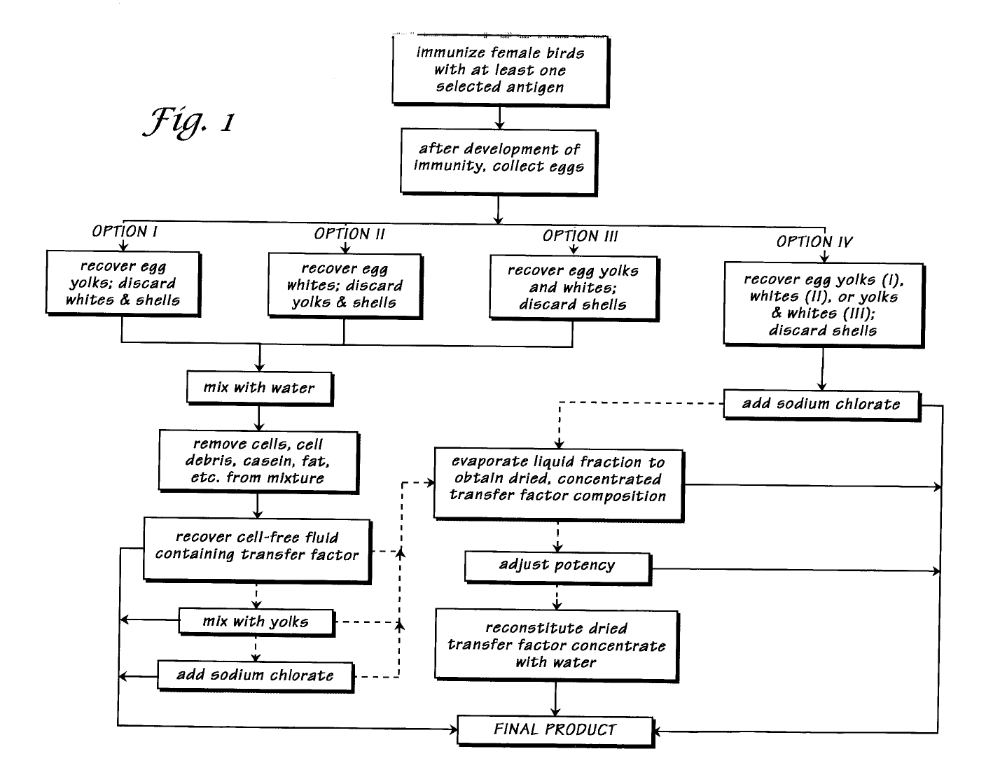

In accordance with a preferred embodiment of the invention, transfer factor is produced from eggs generally as shown in FIG. 1 (alternative or optional process steps for Options I-IV are indicated by dotted lines). The process includes the following steps:

1. Adult hens (i.e., female chickens or other suitable female birds) which have been maintained on water and standard food ad libitum are immunized by administering a selected antigen (or antigens) by any convenient technique, such as intraperitoneal, subcutaneous, I.V. or intramuscular injection. For purposes of this detailed description, adult hens are any female avians that are capable of laying eggs. The terms “immunize,” “sensitize,” and “vaccinate” are used to refer to the process of injecting an animal with an antigen; “immunized,” “sensitized,” and “vaccinated” animals have been so treated.

Antigens may include any of a variety of bacteria, viruses, rickettsiae, fungi, protozoa, and associated vaccines, including but not limited to CMV, EBV, HHV₆, HHV₇, HHV₈, hepatitis A, B, and C, HIV, Herpes 1, Herpes 2, Herpes zoster, Lyme disease, various enteric and other pathogens (E. coli, Salmonella, Pseudomonas aeruginosa, Klebsiella pneumoniae, Haemophilis influenzae, Streptococcus, etc.).

2. After immunizing the hens, a sufficient period of time is allowed for them to develop immunity to the antigen(s). Eggs laid after the development of immunity are collected and treated to recover the transfer factor.

The time needed for immunity to develop depends on factors such as the antigen(s) used, the age and variety of chicken, and such other factors as will be evident to those skilled in the art. Chickens typically respond rapidly to immunization, producing high affinity antibodies within thirty (30) days or less; antibody titers can be maintained for months without further boosting. A sufficient degree of immunity for purposes of this invention may develop by approximately twenty (20) days after immunization, more typically by approximately thirty (30) days after immunization. The dosage required depends on the selection of antibody, the weight of the bird, and other factors known in the art, but may be as low as 20 μg or even lower.

Once the hens have developed immunity to the selected antigen(s), the eggs are collected and treated as described in Steps 3-6 below. To maintain their immunity, the hens may be re-immunized at regular intervals, for example, every 30-120 days or thereabouts.

In order to maximize the production of useful amounts of transfer factor, the hens may re-immunized repeatedly to achieve a state of hyperimmunity wherein they produce larger amounts of transfer factor. The optimum immunization schedule depends on the particular variety of chicken, the selected antibodies, the desired degree of immunity, and so forth. In general, immunization schedules for the development of useful degrees of hyperimmunity range from every 5-10 days to every 30 days. However, intervals of different lengths may also be useful.

While not wishing to be bound by theory, it is believed that fertilized eggs may contain more transfer factor than otherwise comparable nonfertilized eggs. Fertilization and the resultant growth of the embryo may stimulate the production of transfer factor due to the onset of mitosis and related cellular processes.

3. The eggs are collected and washed, and the yolks separated from the whites by any convenient technique (Option I). All procedures are carried out under industry-standard good manufacturing practices. After separation, the yolks are pooled and treated to substantially remove cells, cell debris, casein, and fat by any of a variety of techniques. As noted above, the transfer factor is found primarily in the egg yolk. However, transfer factor found in the albumnin-containing whites may be recovered by a similar process if desired (Option II). Alternatively, the yolks and whites are combined (Options III and IV).

A typical chicken egg contains approximately 12-18 ml of yolk; thus, several hundred milliliters of transfer factor-containing yolks can be recovered from each hen for each month of egg-laying. The amount of yolk recovered from the egg depends on the selected bird (chicken or other avian), the age and weight of the bird, and other factors that will be evident to those of ordinary skill in the art. As will be evident, similar considerations apply to the amount of egg white that can be recovered for use with the invention.

4. By way of example, the separated yolks are passed through a sterile, stainless steel screen. Sterile purified water is added to the yolks (Option I), the whites (Option II), or the combined yolks and whites (Option III) to prepare a suspension, which is centrifuged to separate cells and cell debris, casein, fat, etc., producing a substantially cell-free supernatant fluid containing transfer factor. The solid material (cells, cell debris, etc.) is discarded, and the supernatant is recovered.

5. Alternatively, the cells of the suspension are disrupted by repeated freezing and thawing or by treatment with a suitable chemical agent. When lysis is substantially complete, the resulting mixture is treated to separate the transfer factor-containing fluid from the solids.

Separation may be accomplished through any of a number of techniques, including but not necessarily limited to filtration, centrifugation, dialysis, high performance liquid chromatography (HPLC), and so forth. For example, the supernatant may be passed through a semi-permeable membrane which does not allow the passage of molecules above a certain molecular weight, typically 10,000 or thereabouts. Alternatively, it may be centrifuged to separate the fluid from the solid fraction.

If desired, the transfer factor may be further concentrated, purified, or both. Separation, concentration and purification methods include, but are not necessarily limited to the following: centrifugation, extraction, precipitation, ultrafiltration, dialysis, chromatography, and lyophilization.

6. If desired, the resulting fluid can be dried to reduce its volume, and reconstituted with purified sterile water for use.

Batches of supernatant produced according to the invention can be pooled and treated to obtain a product having a uniform concentration of transfer factor. Transfer factor dosage is usually expressed in terms of units correlated to the quantity of dialysate obtained upon extraction from a given quantity of lymphocytes: a dosage of 1×10⁸ lymphocyte equivalents represents the quantity of transfer factor obtained by isolation from 1×10⁸ lymphocytes obtained from a donor animal. Similarly, transfer factor-containing fluid produced by the above-described process can readily be concentrated or diluted with sterile, purified water to obtain an end product with a selected equivalent dosage. The supernatant obtained as described above can be assayed for transfer factor activity by standard chemical and/or spectroscopic tests. Other suitable tests include, but are not limited to, DLE testing by LiF (leukocyte inhibitor factor), lymphocyte adhesion assay, and traditional footpad/waddle testing on animals.

In another preferred embodiment of the invention, an effective amount of egg yolk is added to the composition produced according to Options I-III. The added yolks, which are preferably in their raw or natural state, are believed to help preserve the activity of transfer factor when it passes through the stomach, resulting in higher bioactivity in the lower digestive tract.

More preferably, an effective amount of sodium chlorate or other suitable compound is added to the above-described compositions. Treatment with sodium chlorate reduces the burden of pathogens such as salmonella and E. coli in the yolks, since these microbes catalyze the intracellular reduction of chlorate to a product which is lethal to the microbes.

An effective amount of sodium chlorate may also be administered to the hens before harvesting the eggs. Such treatment is believed to reduce the bioburden of salmonella (and possibly other pathogens as well) in the eggs laid after such treatment. Sodium chlorate is harmless when ingested in low to moderate amounts, but is converted to sodium chlorite in the digestive tract. Because sodium chlorite is known to be effective against salmonella and E. coli, it has been suggested that cattle, swine, and chickens be treated with sodium chlorate prior to slaughtering.

In still another preferred embodiment of the invention, shown as Option IV, an effective amount of sodium chlorate or other suitable compound is added to the egg yolks and whites, or alternatively to just the yolks or the whites. The resulting composition may be freeze dried or otherwise treated to obtain a dried, concentrated transfer-containing composition.

If desired, the composition may be reconstituted with water or other suitable liquid, and the potency adjusted so as to maintain uniformity between batches.

The operation of the present invention is illustrated by the following non-limiting examples.

### EXAMPLE 1

A group of four-month-old Rhode Island Red hens were inoculated subcutaneously with a selected antigen. Each hen received 200-500 μg of the antigen in a 0.5-1.0 cc volume, which was injected subcutaneously into the breast muscle. In order to simulate a production facility, the hens were housed according to poultry industry standards, and maintained on water and standard food ad libitum.

After two weeks, four randomly-selected hens were subjected to a delayed-type hypersensitivity assay. Results confirmed that the tested hens had developed immunity to the antigen.

Eggs laid by the hens were collected for a period of ten days and stored at refrigerator temperatures. At the end of the collection period, the egg yolks were separated from the whites, pooled, and treated as described above.

The pooled egg yolks were mixed with an equal volume of sterile, purified water to produce a suspension, which was centrifuged as described above. The substantially cell-free supernatant fluid was decanted, and the solid fraction (containing cells, cell debris, etc.) was discarded. The supernatant was passed through a sterile, stainless steel screen. Assays confirmed that the ultraviolet (UV) spectrum of the resulting product was similar to that of transfer factor extracted from peripheral blood lymphocytes.

### EXAMPLE 2

Three patients with antibiotic-resistant Candida albicans and Clostridium infections were treated with a composition according to the present invention.

Twelve hens were immunized with several different strains of Clostridium difficile, Candida albicans, and Candida parapsilosis obtained from the American Type Culture Collection (ATCC). Eggs were collected from day 25 following immunization through day 60. The egg yolks were separated, purified, and filtered to eliminate potential salmonella bioburden, then lyophilized to a powder.

The patients received approximately 600 mg/day of the transfer factor-containing powder, split into two doses (morning and evening). At 35 and 40 days after the start of treatment, all three patients tested negative for the presence of Clostridium and Candida. The patients have remained free of infection on a reduced prophylactic dose of approximately 15-60 mg/day or thereabouts of the powder; however, dosages outside this range may also be useful.

The composition produced by the above-described process may be incorporated into edible products, including nutraceuticals, dietary supplements, and pharmaceutical or veterinary compositions for the prevention or treatment of a disease associated with the selected antigen(s). It may also be administered parenterally or via subcutaneous or intramuscular injection. Topical compositions containing the transfer factor may include one or more constituents such as nontoxic carriers, cleansing agents, humectants, emollients, penetrants such as DMSO, stabilizers, biocompatible perfumes and coloring agents, and other constituents known in the art. The composition may also be useful as a general immune system toner or optimizer, since administration of transfer factor is known to stimulate sluggish or compromised immune systems, and, conversely, suppress overly-active immune systems. Transfer factor can help confer immunity against many different antigens and the diseases associated therewith. For example, transfer factor is believed to be effective in treating conditions such as septicemia, sinusitis, influenza, infections due to various enteric pathogens, diabetes, bronchitis, autism, infertility, the common cold, herpes, bronchitis, measles, mumps, systemic lupus erythematosus (SLE) fibromyalgia, chronic fatigue syndrome, amytrophic lateral sclerosis, multiple sclerosis, rheumatoid arthritis, and cancer.

With respect to the above description of the invention, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing description is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention. Thus, it will be apparent to those skilled in the art that many changes and substitutions can be made to the preferred embodiment herein described without departing from the spirit and scope of the present invention as defined by the appended claims.

What is claimed is:

1. A process for producing transfer factor, said process comprising the steps of
immunizing a female bird with a sufficient quantity of at least one selected antigen so that said bird develops immunity to said at least one antigen;
after said bird develops immunity to said at least one antigen, collecting eggs laid by said bird; and
treating said eggs to recover transfer factor therefrom.

2. The process as recited in claim 1, wherein said treating step further comprises the steps of
separating the egg yolks from said eggs;
mixing said egg yolks with water to produce a suspension;
removing cells and cell debris from said suspension to produce a fluid containing at least some of said transfer factor; and
recovering said fluid.

3. The process as recited in claim 1, wherein said treating step further comprises the steps of:
separating the egg whites from said eggs;
mixing said egg whites with water to produce a suspension;
removing cells and cell debris from said suspension to produce a fluid containing at least some of said transfer factor; and
recovering said fluid.

4. The process as recited in claim 1, wherein said treating step further comprises the steps of:
mixing the egg whites and egg yolks with water to produce a suspension;
removing cells and cell debris from said suspension to produce a fluid containing at least some of said transfer factor; and
recovering said fluid.

5. The process as recited in claim 1, wherein said transfer factor is contained in a fluid recovered from said eggs, further comprising the step of evaporating said fluid.

6. The process as recited in claim 1, further comprising the step of adding an effective amount of natural egg yolk to said composition.

7. The process as recited in claim 1, further comprising the step of adding an effective amount of sodium chlorate to said composition.

8. The process as recited in claim 1, further comprising the initial step of administering an effective dose of sodium chlorate to said birds.

9. The process as recited in claim 1, wherein said bird is of the family Phasianidae.

10. A transfer factor composition, said composition made by a process comprising the steps of:
immunizing a female bird with a sufficient quantity of at least one selected antigen so that said bird develops immunity to said at least one antigen;
after said bird develops immunity to said at least one antigen, collecting eggs laid by said bird; and
treating said eggs to recover transfer factor therefrom.

11. The composition as recited in claim 10, wherein said treating step further comprises the steps of:
separating the egg yolks from said eggs;
mixing said egg yolks with water to produce a suspension,
removing cells and cell debris from said suspension to produce a fluid containing at least some of said transfer factor; and
recovering said fluid.

12. The composition as recited in claim 10, wherein said treating step further comprises the steps of:
separating the egg whites from said eggs;
mixing said egg whites with water to produce a suspension;
removing cells and cell debris from said suspension to produce a fluid containing at least some of said transfer factor; and
recovering said fluid.

13. The composition as recited in claim 10, wherein said treating step further comprises the steps of:
mixing the egg whites and egg yolks with water to produce a suspension;
removing cells and cell debris from said suspension to produce a fluid containing at least some of said transfer factor; and
recovering said fluid.

14. The composition as recited in claim 10, wherein said transfer factor is contained in a fluid recovered from said eggs, further comprising the step of evaporating said fluid.

15. The composition as recited in claim 10, further comprising the step of adding an effective amount of natural egg yolk to said composition.

16. The composition as recited in claim 10, further comprising the step of adding an effective amount of sodium chlorate to said composition.

17. The composition as recited in claim 10, further comprising the initial step of administering an effective dose of sodium chlorate to said birds.

18. The composition as recited in claim 10, further comprising at least one added constituent added to said fluid, said constituent selected from the group consisting of edible and injectable constituents.

19. A method for treating the immune system of an animal, said method comprising the step of administering to said animal a composition containing transfer factor, said composition made by a process comprising the steps of:
immunizing a female bird with a sufficient quantity of at least one selected antigen so that said bird develops immunity to said at least one antigen;
after said bird develops immunity to said at least one antigen, collecting eggs laid by said bird; and
adding said transfer factor to an edible or an injectable constituent.

20. The method as recited in claim 19, wherein said bird is gallinaceous.
